(19) United States
(12) Patent Application Publication
(10) Pub. No.: US 2019/0032125 A1
(43) Pub. Date: Jan. 31, 2019

(54) METHOD OF DETECTING CHROMOSOMAL ABNORMALITIES

(71) Applicant: EONE DIAGNOMICS GENOME CENTER CO., LTD, Incheon (KR)

(72) Inventors: Chang Hyuk KWON, Incheon (KR); Seon Young YUN, Incheon (KR); Min Seob LEE, Incheon (KR)

(73) Assignee: EONE DIAGNOMICS GENOME CENTER CO., LTD, Incheon (KR)

(21) Appl. No.: 16/071,537

(22) PCT Filed: Jan. 20, 2017
(86) PCT No.: PCT/KR2017/000741

§ 371 Date: Jul. 20, 2018

(30) Foreign Application Priority Data
Jan. 20, 2016 (KR) 10-2016-0007181

(51) Int. Cl. C12Q 1/6869; G06F 19/24; G06F 19/22; G06F 17/12; C12Q 1/6883; G06F 17/18

(52) U.S. Cl. CPC C12Q 1/6869; G06F 19/24; G06F 19/22; C12Q 2600/156; C12Q 1/6883; G06F 17/18; G06F 17/12

(57) ABSTRACT

A Method is provided for determining chromosome abnormalities. The method includes sequencing next-generation sequencing (NGS) sequence data regardless of an NGS analysis platform, determining male or female by extracting a unique-read from the sequenced sequence data, and setting a threshold line using initial learning by linear discriminant analysis (LDA) of existing data, thereby being applied for both autosomes and sex-chromosomes, and improving accuracy and sensitivity as the number of diagnoses increases.

### BACKGROUND OF THE INVENTION

### Field of the Invention

The present invention relates to a method for determining chromosome abnormalities, and more particularly, to a new method for determining chromosome abnormalities, including sequencing next-generation sequencing (NGS) sequence data regardless of an NGS analysis platform, determining male or female by extracting a unique-read from the sequenced sequence data, and setting a threshold line using initial learning by linear discriminant analysis (LDA) of existing data, thereby being applied for both autosomes and sex-chromosomes, and improving accuracy and sensitivity as the number of diagnoses increases.

### Related Art

‘Prenatal diagnosis’ refers to a process of determining and diagnosing presence or absence of fetal diseases before the birth of the fetus. According to recent statistics, it has been reported that congenital malformed children account for about 3% of all neonates and about 20% of the congenital malformed children are caused by chromosome abnormalities. Specifically, the congenital malformed child which is widely known as Down syndrome corresponds to 26% of the congenital malformed children.

Due to the increased birth rate of malformed children and the development of various prenatal diagnostic devices, interest in prenatal diagnosis is increasing day by day. In particular, in the case where there is an elderly pregnant woman over 35 years of age, there is a pregnant woman with a childbirth history of chromosome abnormalities there is one of the parents having a family history of genetic disease, there is a family history of genetic disease, there is a risk of neural tube defects, and fetal malformation is suspected in maternal serum screening and ultrasonography, the prenatal diagnosis is required.

The prenatal diagnosis method may be largely divided into invasive and noninvasive diagnostic methods. Examples of the invasive diagnostic method include chorionic villi sampling (CVS) performed during 10 and 12 weeks of pregnancy, amniocentesis of analyzing fetal chromosomes by measuring a concentration of AFP in amniotic fluid using immunoassay during 15 to 20 weeks of pregnancy, a cordocentesis method in which fetal blood is extracted directly from the umbilical cord under ultrasound-induced during 18 to 20 weeks of pregnancy, and the like.

However, these invasive diagnostic methods may cause abortion, illness or malformation by impacting the fetus during the examination process. Methods of securing fetal material by amniocentesis or chorionic villus sampling may be invasive, and non-negligible risks to pregnancy may be caused even by skilled clinicians. In current practice, these invasive diagnostic methods are generally used when there is a sign that the probability of down syndrome fetal pregnancy due to maternal age or pre-screening through biochemical testing or ultrasound examination.

Noninvasive diagnostic methods have been developed to overcome the problems of these invasive diagnostic methods. For example, the pre-embryonic genetic diagnosis method is a technique for selecting embryos without preimplantation intrauterine genetic defects using molecular genetics or cytogenetic techniques used in in-vitro fertilization. In addition, a quantitative-fluorescent PCR (QF-PCR) fluorescence assay for rapid diagnosing chromosome aneuploidy is a quick screening test method of measuring and analyzing an amount of amplified DNA labeled with fluorescence by a DNA automatic sequence analyzer after amplifying short tandem repeats (STR) of the DNA that are specific for each chromosome and labeled with the fluorescence by a multiplex PCR method. In addition, in order to find a copy number change, a chromosome microarray (CMA) method is known for collecting and inspecting DNA sequences mapped onto a glass slide.

Meanwhile, with the development of a sequencing technology, as it becomes possible to decode large-scale genome information, genome analysis methods based on a next-generation sequencing (NGS) technology are utilized even in the field of prenatal diagnosis. In particular, it is known that cellular free DNA in the plasma of pregnant women contains components of the fetal origin (Lo et al., 1997, Lancet 350, 485-487), and in cell free plasma DNA (hereinafter, referred to as “serum DNA”), 5% to 20% originates from the fetal, and the remainder is often formed of short DNA molecules (80 to 200 bp) of the maternal (Birch et al., 2005, ClinChem 51, 312-320; Fan et al., 2010, ClinChem 56, 1279-1286).

Prenatal diagnosis methods for isolating the fetal cells from the maternal blood and analyzing chromosomes using these facts are known. In general, since the conditions having chromosome aneuploidy which is caused by excess chromosomes or chromosome defects produce an imbalance of a fetal DNA molecule cluster in the detectable maternal free plasma DNA, methods of analyzing chromosome abnormalities using the same have been developed.

In principle, if the cellular free DNA in the plasma is not diluted by the maternal component, the excess chromosome that causes characteristics of T21 is expected to produce more than 50% DNA molecules derived from the chromosome as compared to normal pregnancy. However, when considering a typical value of 10% for the components of the cellular free plasma DNA of fetal origin, the resulting imbalance is only 5%, or expected to be a relative increase in the number of chromosome 21-derived fragments at a value of 1.05 compared to 1.00 for normal pregnancy. In situations where the fetal component of plasma DNA is smaller or larger than the 10% value, the imbalance in the number of chromosome 21-derived molecules within the cluster of molecules in the maternal plasma is correspondingly smaller or larger.

Thus, the basis of this non-invasive diagnostic test is obtaining nucleotide sequence data for DNA molecules from the maternal plasma (‘DNA sequence analysis’). After partial or complete nucleotide sequence information is obtained from individual DNA molecules, bioinformatics techniques need to be applied to assign individual molecules to the chromosome originated by the molecules most simply by comparison with the reference human genome(s).

Considering that bioinformatic methods can be reliably applied to obtain some nucleotide sequence data for a sufficiently large number of plasma DNAs and assign a sufficiently large number of genes to its chromosome origin, statistical methods may be applied to determine the presence or absence of chromosome imbalances in a cluster of plasma DNA molecules while retaining statistical reliability.

Up to now, in this diagnostic method, in order to obtain a sequence having a length enough to be assigned to a chromosome origin thereof, a large-scale parallel DNA sequencing technique which generates high-quality sequence data that is relatively error-free (known as next-generation sequencing or second-generation sequencing) was used.

This specific automated sequencing device generates sequence data that is substantially less than that normally required for general genomic sequencing. The sequence data generated as such is characterized by frequent errors. Types of these errors are various, but ‘insertion-deletion (indel)’ is most common and is an error caused by a sequencing device which delivers an inaccurate excess base (insertion) or a deleted base. In addition, it is difficult to effectively sequence a short homopolymer run (i.e., a run of several identical bases). In addition, the sequencing error may also include “mismatch” in which the base is incorrectly assigned, and tends to indicate various errors.

In addition, such a massive parallel sequencing has disadvantages in that the performed sequencing requires much time and is performed with high quality in a full-service genome sequencer, mainly Illumina HiSeq, which generates very large data requiring expensive bioinformatics. In addition, the method of performing the specific analysis varies depending on a kind of full-service genome sequencer, and the execution time and the analysis process may take several weeks as a whole.

### SUMMARY OF THE INVENTION

In order to solve the problems of the related art as described above, the present invention is not limited to a sequencing method by a specific automatic sequencer and a normalization method thereof in the related art, and an object of the present invention is to provide a new method for determining chromosome abnormalities which are able to use generated sequence information and be applied for both autosomes and sex-chromosomes.

In order to solve the above objects, an aspect of the present invention provides a method for determining chromosome abnormalities including:

a first step of extracting a unique read from sequenced sequencing data of a target chromosome;

a second step of setting a threshold line for determining chromosome aneuploidy by linear discriminant analysis (LDA) by dividing and labeling normality and aneuploidy of chromosome data pre-verified for the normality and aneuploidy; and

a third step of determining whether there is aneuploidy of the unique read-target chromosome gene extracted in the first step by the threshold line set in the second step.

In the method of determining the chromosome abnormalities according to the present invention, in the second step of setting the threshold line for determining the aneuploidy, the normality and the aneuploidy of the chromosome data pre-verified for the normality and the aneuploidy are divided and labeled to be initially learned by the LDA and a minimum value of the aneuploidy chromosome data among the pre-verified chromosome data is set as the threshold value.

In the method of determining the chromosome abnormalities according to the present invention, the LDA technique refers to a linear discriminant analysis method and refers to a method of setting an initial threshold value by analyzing the pre-verified chromosome data and setting a minimum value of the aneuploidy chromosome data as the threshold line by additionally analyzing the accumulated samples.

In the method of determining the chromosome abnormalities according to the present invention, in the step of determining whether there is the aneuploidy of the new target chromosome gene according to the criteria set by the LDA method, the presence or absence of chromosome abnormalities is determined by setting a range of a normal sample from the pre-verified chromosome data and setting a minimum value of the aneuploidic data as the threshold value.

In the method for determining the chromosome abnormalities according to the present invention, in the step of extracting the unique read from the target chromosome, the unique read which is divided into a 90 kb bin region and has the GC content of 0.35 to 0.55 or less is extracted.

The method for determining the chromosome abnormalities according to the present invention further includes, after the first step, a 1-1 step of calculating UR(x) % (percentage of reads uniquely matched to a chromosome X) and UR(y) % (percentage of reads uniquely matched to a chromosome Y) represented by the following Formulas from the extracted unique read;

UR(x) %=Number of reads of chromosome X (chrX)/total number of (autosomes) reads×100

UR(y) %=Number of reads of chromosome Y (chrY)/total number of (autosomes) reads×100

a 1-2 step of discriminating gender from the UR(x) % and the UR(y) %; and

a 1-3 step of discriminating gender from the number of reads of the region matched to a Y-specific region in the step of discriminating the gender from the UR(x) % and the UR(y) %.

In the method for determining the chromosome abnormalities according to the present invention, in the step of discriminating the gender from the UR(x) % and the UR(y) %, the gender is discriminated from the number of reads in the region (Table 1) matched to the Y-specific region which selects only a pure chrY region by selecting a pseudoautosomal region by comparing chrX and chrY to remove a chrX region.

In the method for determining the chromosome abnormalities according to the present invention, the chromosome is at least one chromosome selected from the group consisting of chromosome 13, chromosome 18, chromosome 21, chromosome 3, chromosome 7, and chromosome 12, a chromosome X or a chromosome Y.

In the method for determining the chromosome abnormalities according to the present invention, it is possible to be extended to whole autosomes when the autosomes are targeted, and in the method for determining the chromosome abnormalities according to the present invention, examples of the chromosome abnormalities include:

down syndrome (Trisomy 21), Edward syndrome (Trisomy 18), Patau syndrome (Trisomy 13), Trisomy 9, Warkany syndrome (Trisomy 8), Cat Eye syndrome (4 copies of chromosome 22), Trisomy 22, and Trisomy 16.

Additionally or alternatively, the detection of an abnormality of genes, chromosomes, or some of chromosomes, and the copy number may include detection and/or diagnosis of a condition selected from the group consisting of: Wolf-Hirschhorn syndrome (4p−), Cri du chat syndrome (5p−), Williams-Beuren syndrome (7−), Jacobsen syndrome (11−), Miller-Dieker syndrome (17−), Smith-Magenis syndrome (17−), 22ql 1.2 Deletion syndrome (also known as Velocardiofacial syndrome, DiGeorge syndrome, conotruncal anomaly face syndrome, congenital thymic dysplasia, and Strong's syndrome), Angelman syndrome (15−), and Prader-Willi syndrome (15−).

Additionally or alternatively, the detection of the abnormality of the chromosome copy number may include detection and/or diagnosis of a condition selected from the group consisting of Turner syndrome (Ullrich-Turner syndrome or single chromosome X), Klinefelter syndrome, 47,XXY or XXY syndrome, 48,XXXY syndrome, 49,XXXXY syndrome, Triple X syndrome, XXXX syndrome (also referred to as tetrasomic X, quadruple X, or 48,XXXX), XXXXX syndrome (also referred to as pentasomic X or 49,XXXXX), and XYY syndrome.

In the method for determining the chromosome abnormalities according to the present invention, since the threshold line for determining the chromosome aneuploidy is set by the LDA method from the existing sequenced data, the more an amount of sequenced data to be used, the higher accuracy and sensitivity of the determination, and as a result, the accuracy and sensitivity of the determination may be continuously improved at the time of performing the method many times while the data is continuously accumulated.

That is, in the method for determining the chromosome abnormalities according to the present invention, it is possible to perform the first to third steps for determining the chromosome abnormalities N times while continuously adding sequenced data sequences. When a chromosome data used at the time of the N−1-th determination is referred to as Dn−1 and a chromosome data used at the time of the N-th determination is referred to as Dn, the determination of the aneuploidy for the chromosome data Dn used at the time of the N-th determination is a threshold value derived from the chromosome data Dn−1 used at the time of the N−1-th determination.

The threshold value is affected by a specific algorithm, but a value close to the aneuploidy is set to one value or the threshold value is set to two values, and as a result, the determination may also be flexibly improved.

In the method for determining the chromosome abnormalities according to the present invention, the sequenced sequence data is obtained by a next-generation sequencing platform. It will be understood by those of ordinary skill in the art that the method for obtaining the sequence data according to the present invention is not limited to any specific technique.

The sequencing platform was discussed and reviewed from literatures [Loman et al. (2012) Nature Biotechnology 30(5), 434-439]; [Quail et al. (2012) BMC Genomics 13, 341]; [Liu et al. (2012) Journal of Biomedicine and Biotechnology 2012, 1-11]; and Meldrum et al. (2011) ClinBiochem Rev. 32(4): 177-195]; and the sequencing platform reviewed from the literatures is included in the present application by reference.

In the method for determining the chromosome abnormalities according to the present invention, the next-generation sequencing platform is selected from a Roche 454 (i.e., Roche 454 GS FLX), a SOLiD system from Applied Biosystems (i.e., SOLiDv4), GAIIx, HiSeq 2500 and MiSeq sequencers from Illumina, Proton and S5 sequencers of Ion Torrent semiconductor sequencing platforms from Life Technologies, PacBio RS from Pacific Biosciences, and 3730xl from Sanger.

In the method for determining the chromosome abnormalities according to the present invention, the sequenced sequence data is obtained by a sequencing platform including the use of a polymerase chain reaction.

In the method for determining the chromosome abnormalities according to the present invention, the sequenced sequence data is obtained by a sequencing platform including the use of sequencing by synthesis.

In the method for determining the chromosome abnormalities according to the present invention, the sequenced sequence data is obtained by a sequencing platform including the use of ions, for example, hydrogen ion release.

In the method for determining the chromosome abnormalities according to the present invention, the sequenced sequence data is obtained by a sequencing platform including the use of a semiconductor-based sequencing method. The advantage of the semiconductor-based sequencing method is that the manufacturing cost of devices, chips and reagents is low, the sequencing process is rapid (despite off-set by emPCR) and the system can be extended, but it may be somewhat limited to a bead size used in the emPCR.

In the method for determining the chromosome abnormalities according to the present invention, the sequenced sequence data is obtained by a sequencing platform including the use of a nanopore-based sequencing method. The nanopore-based method includes the use of organic-type nanopores that imitate conditions of a cell membrane and a protein channel of living cells, like a technique used by, for example, Oxford Nanopore Technologies (e.g., Literature [Branton D, Bayley H, et al. (2008). Nature Biotechnology 26 (10), 1146-1153]).

In the method for determining the chromosome abnormalities according to the present invention, the sequenced sequence data is obtained by an Ion Torrent platform from Life Technologies or MiSeq from Illumina. A sequencing technique by synthesis of Illumina (SBS) is currently successful, and a next-generation sequencing platform which is widely adopted worldwide. A TruSeq technique supports large-scale parallel sequencing using an exclusive reversible terminator-based method that enables its detection when a single base is included in a growing DNA strand. A fluorescence-labeled terminator is imaged by adding each dNTP and then cleaved to allow introduction of the next base. Since all four reversible terminator-binding dNTPs exist during each sequencing cycle, natural competition minimizes introduction bias.

In the method for determining the chromosome abnormalities according to the present invention, the sequenced sequence data is obtained by an Ion Torrent personal genome machine (Ion Torrent PGM) from Life Technologies.

In the method for determining the chromosome abnormalities according to the present invention, the sequenced sequence data is obtained by an Ion Torrent platform from Life Technologies, for example, Ion Proton and S5 having PI or PII chips, and multiplex capable iteration based on additional derivative devices and components thereof.

In an additional embodiment, the next-generation sequencing platform is a personal genome machine (PGM), which is the Ion Torrent personal genome machine from Life Technologies. The Ion Torrent device uses a strategy similar to sequencing by synthesis (SBS), but detects signals by the release of hydrogen ions according to the activity of a DNA polymerase during the nucleotide introduction. Essentially, the Ion Torrent chip is a very sensitive pH meter. Each ion chip includes millions of ion-sensitive field effect transistor (ISFET) sensors that allow simultaneous detection of multiple sequencing reactions. The use of the ISFET device is well known to those skilled in the art and may be performed within a range of a technique which may be used to obtain the sequence data required by the method of the present invention. (Prodromakis et al. (2010) IEEE Electron Device Letters 31(9), 1053-1055; Purushothaman et al. (2006) Sensors and Actuators B 114, 964-968; Toumazou and Cass (2007) Phil. Trans. R. Soc. B, 362, 1321-1328; WO 2008/107014 (from DNA Electronics Ltd); WO 2003/073088 (from Toumazou); US 2010/0159461 (from DNA Electronics Ltd); each sequencing method is included in the present application by reference).

In the method for determining the chromosome abnormalities according to the present invention, the sequenced sequence data is normalized or not. That is, the method for determining the chromosome abnormalities according to the present invention is not limited to the sequencing method, and may determine the chromosome abnormalities even in the case of performing or not standardization and normalization of the sequenced sequence data.

### Advantageous Effects

The method for determining the chromosome abnormalities according to the present invention is not limited to the sequencing method and the normalization method thereof by a specific automatic sequencing device in the related art. The method can be usefully used for prenatal diagnosis by using the generated sequence information, being applied to autosomes and sex-chromosomes, and early determining presence or absence of malformation due to abnormality of the number of fetal autosomes and sex-chromosomes based on a commercial application of a non-invasive method because as the number of diagnoses increases, accuracy and sensitivity increase.

In the method according to the present invention, when many sequencing data and abnormality determination data therefor are accumulated, it is possible to set a precise threshold line by a linear discriminant analysis (LDA) method, thereby obtaining the sensitivity much higher than that of the conventional method.

### BRIEF DESCRIPTION OF THE DRAWINGS

FIG. 1 is a graph showing an example of determining gender as a Y-specific region by protons with respect to 100 samples using a diagnostic method of the present invention.

FIG. 2 is a graph showing an example of determining gender by a HiSeq platform from Illumina Co., Ltd. with respect to 30 samples using the diagnostic method of the present invention.

FIG. 3 is a graph showing a result of predicting a new sample after learning by performing normalization with QDNAseq using the diagnostic method of the present invention.

FIG. 4 is a graph showing a result of predicting a new sample after learning by performing normalization with HMMcopy using the diagnostic method of the present invention.

FIG. 5 is a graph showing a result of predicting a new sample after learning using only a percentage of X and Y without normalization.

FIG. 6 is a graph showing a result of predicting a new sample after learning by performing normalization with Deeptools using GCBias by using the diagnostic method of the present invention.

FIG. 7 is a graph showing a result of discriminating normality and aneuploidic samples of chromosome 21 using the diagnostic method of the present invention. Here, N is a normal sample, T is an aneuploidic sample, and a red T is a sample in a threshold line.

FIG. 8 is a graph showing a result of discriminating normality and aneuploidic samples of chromosome 18 using the diagnostic method of the present invention. Here, N is a normal sample, R is an aneuploidic sample, and a red R is a sample in a threshold line.

FIG. 9 is a graph showing a result of discriminating normality and aneuploidic samples of chromosome 13 using the diagnostic method of the present invention. Here, N is a normal sample, M is an aneuploidic sample, and a red M is a sample in a threshold line.

FIG. 10 is a graph simultaneously showing the determination of chromosomes 21 and 18 using the diagnostic method of the present invention. Here, a horizontal axis is chr21, a vertical axis is chr18, N is normal, white is aneuploidy 18, and pink is aneuploidy 21.

FIG. 11 is a graph showing a result of determining aneuploidy of chromosome 3 using the diagnostic method of the present invention. In QDNAseq, an average of the normal samples is 7.551 and an average of the aneuploidic samples is 7.615.

FIG. 12 is a graph showing aneuploidic samples of chromosome 7 using the diagnostic method of the present invention.

FIG. 13 is a graph showing aneuploidic samples of chromosome 12 using the diagnostic method of the present invention.

FIGS. 14 to 16 are graphs showing a normal sample and XXY, XYY, XXX, and XO samples to determine chromosome aneuploidy using the diagnostic method of the present invention.

FIG. 15 is a graph for discriminating XXY from XYY.

FIG. 16 is a graph for discriminating XXY from XO.

### DESCRIPTION OF EXEMPLARY EMBODIMENTS

Hereinafter, the present invention will be described in more detail through Examples. These Examples are just to exemplify the present invention, and it is apparent to those skilled in the art that it is not interpreted that the scope of the present invention is not limited to these Examples.

Unless otherwise defined, all technical and scientific terms used in this specification have the same meaning as those commonly understood by those skilled in the art. In general, the nomenclature used and the experimental method described below in this specification is well-known and commonly used in the art.

### EXAMPLE 1

### Discriminating Male or Female by Extracting Unique Read

Plasma was extracted from the blood collected from the mother, and a library was prepared by extracting 30 ng or more of cfDNA from the plasma. And both Life Tech and Illumina were combined with an adapter. Thereafter, pooling was performed by E-gel size selection using Life Tech equipment, bead size selection was performed using Illumina, and sequencing was performed by pooling.

Sequenced fastq files were sorted and PCR duplication was removed to extract unique reads. Only the perfectly matched reads were sorted, and all the regions in the sorted sequence were divided into 90 kb bin regions and reads with a GC content of 0.35 to 0.55 or less were extracted.

A percentage UR(x) % of free reads which are uniquely matched with a chromosome X and a percentage UR(y) % of free reads which are uniquely matched with a chromosome Y represented by the following Formulas were obtained.

−UR(x) %=Number of reads of chromosome X (chrX)/total number of (autosomes) reads×100

−UR(y) %=Number of reads of chromosome Y (chrY)/total number of (autosomes) reads×100

As shown in Table 1 below, a Y-specific region was set, and the number of reads was calculated based on the Y-specific region, and then when the number of reads was less than 2, it was determined as female and when the number of reads was 2 or more, it was determined as male.

In Table 1 below, the Y-specific region is defined as a pure chrY region by removing a chrX region after removing a pseudoautosomal region by comparing chrX and chrY, and the Y-specific region selected as follows. The present invention is characterized in that it is possible to easily discriminate male and female by using a method of counting the number of reads in a region mapped to the Y-specific region.

TABLE 1

| Y-specific region | The same region as X |
|---|---|
| - chrY:1-10000chrY:10001-2649520- | - chrX:60,001-2,699,520=chrY:10,001- |
| chrY:2649521-59034049chrY: 59034050- | 2,649,520- |
| 59373566 | chrX:154,931,044=chrY:59,034,050- |
| | 59,363,566 |

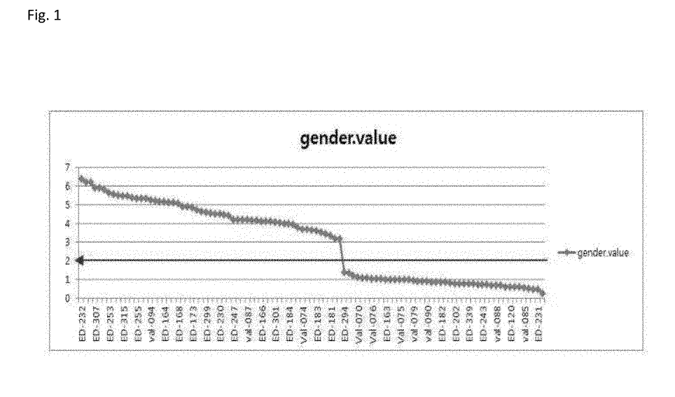

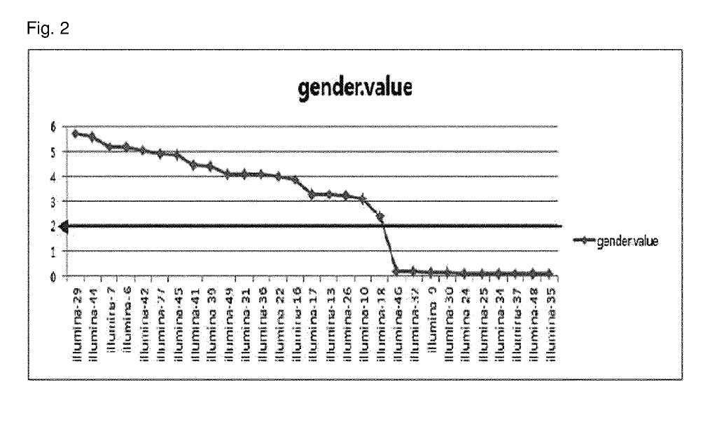

In FIG. 1 showing a case in which gender was measured by performing initial learning using a LDA method according to the present invention with respect to 100 samples using proton and FIG. 2 showing a case in which gender is measured with respect to 30 samples using Illumina, it can be seen that although threshold values determined by the LDA are different in each case, male and female may be discriminated by mutually similar values.

### EXAMPLE 2

### LDA Learning Using Existing Sequencing Data

In the present invention, the data identified by the standard method is initially learned using the LDA method, a minimum value of aneuploidic data is extracted as a threshold value, and normal, aneuploidy, and threshold of a target chromosome may be predicted from this.

Conventional methods such as Z-score and NCV of Illumina are typically used, but various normalization algorithms (QDNAseq, HMMcopy, Deeptools, etc.) for normalizing the entire data using low-depth data have been introduced.

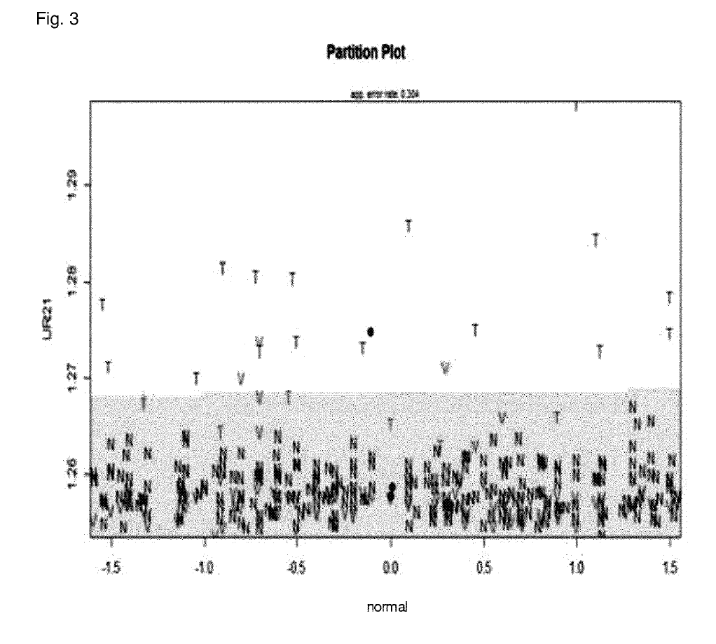

Referring to FIG. 3, which shows the result of normalization of the sequencing data and obtaining a Z-score with a QDNAseq program using loess, it can be seen that 5 red T (Trisomy) samples may be identified, and since normal and aneuploidic samples are discriminated at 1.268, 1,268 is able to be automatically set as a threshold line by the LDA method.

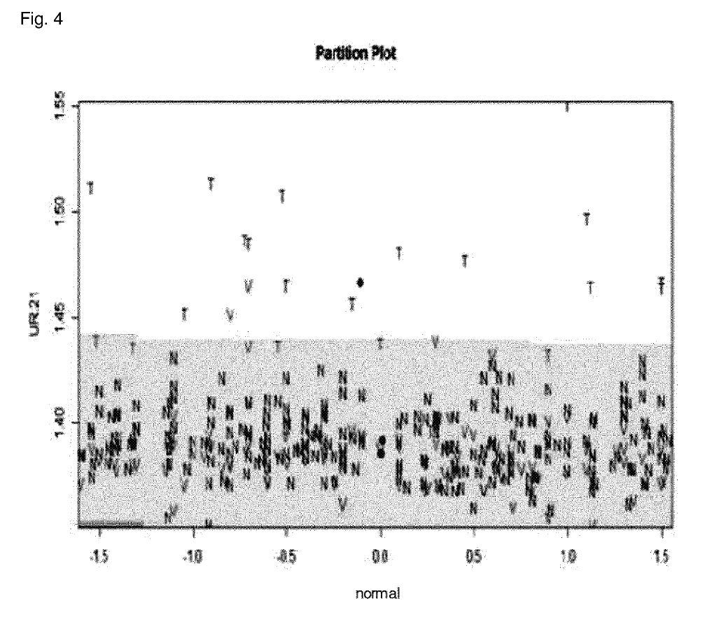

In FIG. 4, which shows the result of normalizing HMMcopy and calculating a Z-score, it can be seen that five red T (Trisomy) samples can be identified and there are two N (normal), but since the normal and aneuploidic samples are clearly discriminated based on 1.44, 1,44 is able to be automatically set as a threshold line by the LDA method.

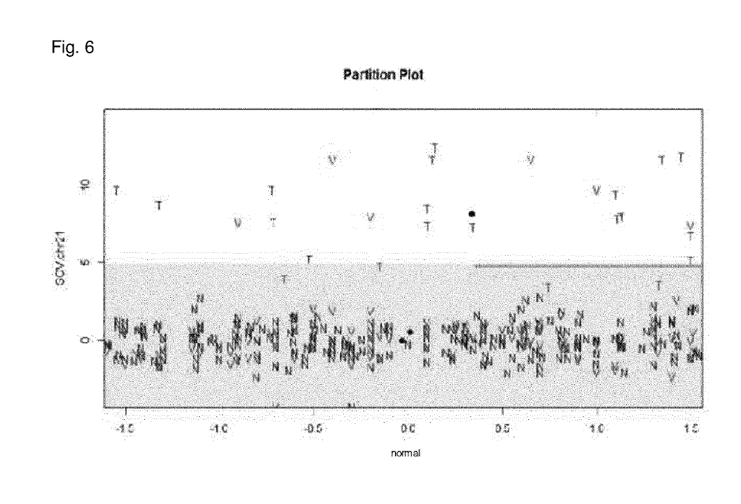

In FIG. 6 which shows a result of normalizing only GCBias, it can be seen that since the normal and aneuploidic samples are clearly discriminated based on 5, 5 is able to be automatically set as a threshold line by the LDA method.

In addition, in the method for determining the chromosome abnormalities of the present invention, it is possible to determine chromosome abnormalities without performing a separate normalization process with respect to the sequenced data regardless of a specific platform.

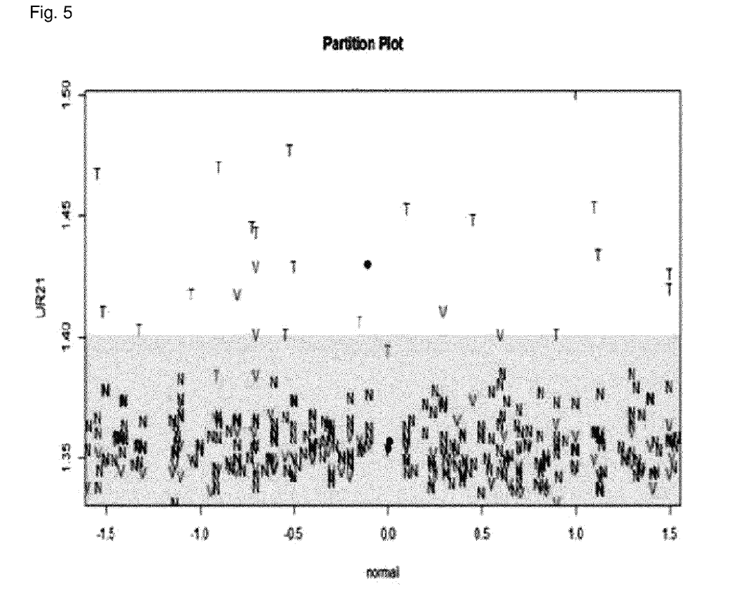

In FIG. 5, it can be seen that data is learned only by the percentages of UR.X and UR.Y without performing normalization after performing basic sequencing, and then even if a value (red V) of a new sample value is inserted, a normal black sample N and a black aneuploidic sample T are clearly discriminated based on 1.4.

In FIG. 5, it can be seen that since there are only two red T included in the threshold line, in the case of the method for determining the chromosome abnormalities by the LDA technique according to the present invention, a normal sample and an aneuploidic sample may be clearly discriminated while performing only a simple sorting sequence.

From this, in the case of the method for determining the chromosome abnormalities by the LDA technique according to the present invention, it can be seen that the same result can be obtained without using the known normalization algorithm or the Z-score.

### EXAMPLE 3

### Determination of Aneuploidy of Autosomes

### EXAMPLE 3-1

### Determination of Aneuploidy of Chromosomes 21, 18, and 13

The cases of chr21, chr18 and chr13 are discriminated from the data confirmed by the existing standard method of Example 2, and a minimum value of the aneuploidic data is extracted as a threshold value using the LDA method for each of the chr21, chr18 and chr13 data, thereby predicting and determining normal, aneuploidy, and threshold.

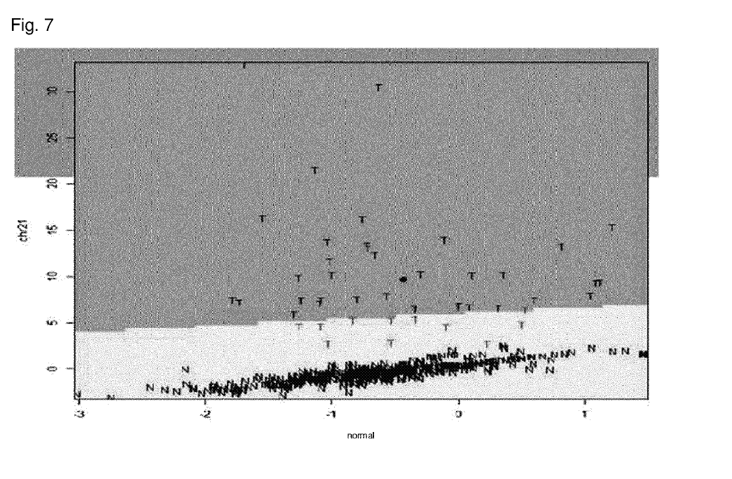

In the method of determining the chromosome abnormalities according to the present invention, that is, by performing the sorting sequence using existing data, performing normalization, and then setting a minimum value of the aneuploidic data selected by the LDA method as a threshold value, results of determining aneuploidy of chromosomes chr21, chr18, and chr13 based on the threshold value were shown in FIGS. 7, 8, and 9.

In FIG. 7, it can be seen that it is possible to determine clearly aneuploidy based on the threshold value of 4 in the case of chr21, and to clearly discriminate a normal (N) sample and an aneuploidy (T) sample from a threshold line based on a red T (aneuploidy) sample.

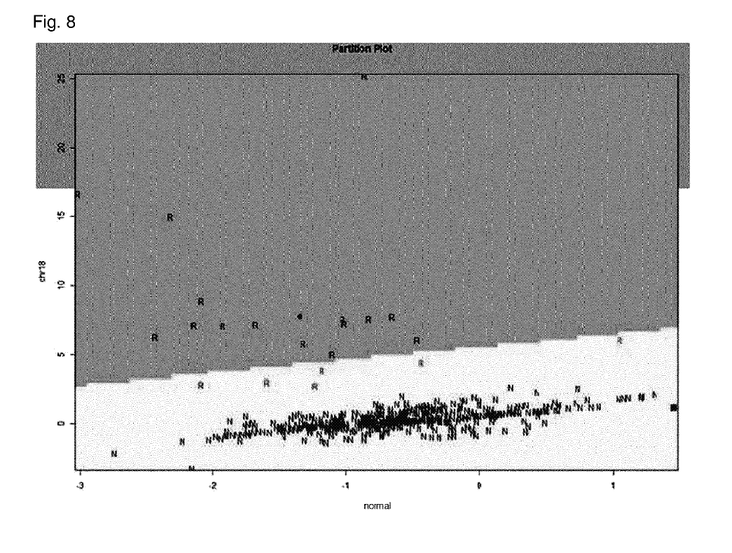

In FIG. 8, it can be seen that it is possible to determine clearly aneuploidy based on the threshold value of 2.5 in the case of chrt18, and to clearly discriminate a normal (N) sample and an aneuploidy (T) sample from a threshold line based on a red R (aneuploidy) sample.

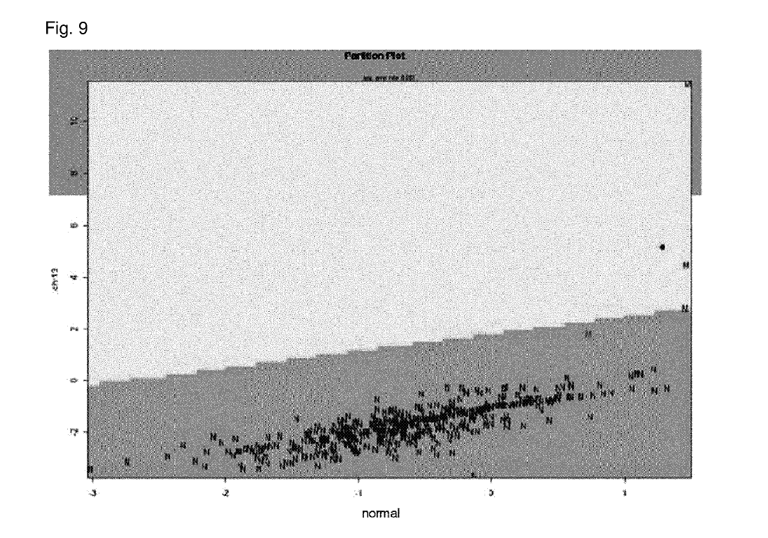

In FIG. 9, it can be seen that it is possible to determine clearly aneuploidy based on the threshold value of 1.5 in the case of chrt13, and to clearly discriminate a normal (N) sample and an aneuploidy (T) sample from a threshold line based on a red M (aneuploidy) sample.

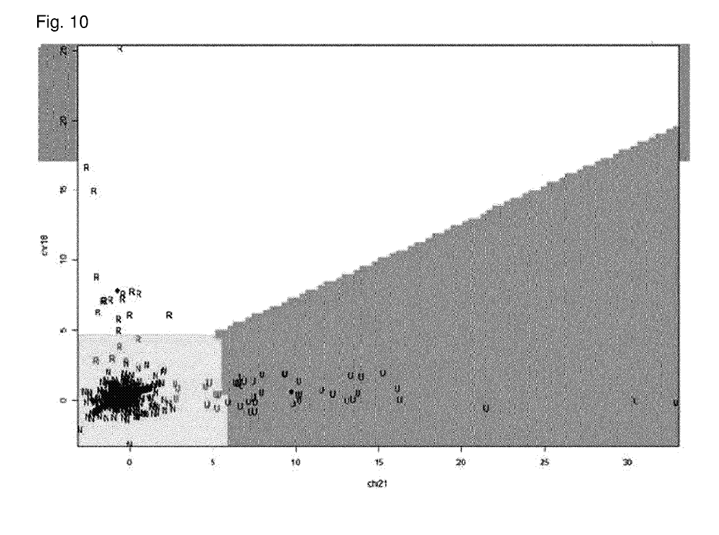

Also, as shown in FIG. 10, it can be confirmed that in the method for determining the chromosome abnormalities of the present invention, chr21 and chr18 may easily discriminate the samples showing aneuploidy at the same time.

### EXAMPLE 3-2

### Possibility of Extension of Autosomal Region

It has been confirmed that the method for determining the chromosome abnormalities of the present invention is able to be applied not only to the most well-known chr13, chr18, and chr21, but also to other autosome abnormalities.

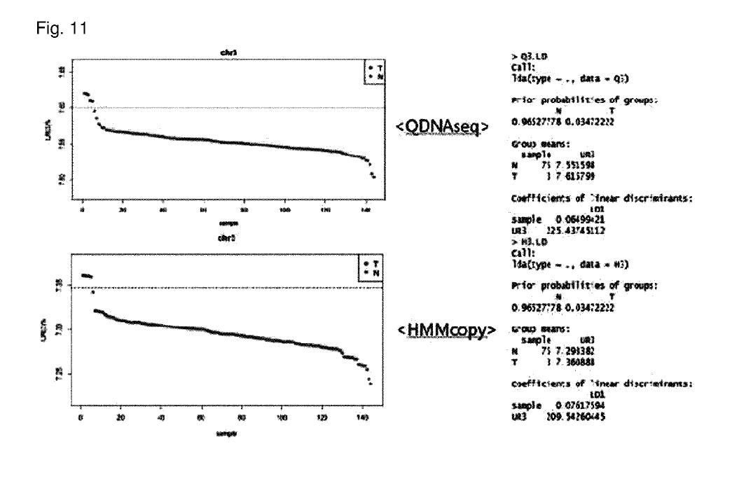

First, Normalization was performed by a conventionally used method from the three chromosome sequencing data of chr3, chr7, and chr12. And z-score was calculated by using the number of reads, and then results are shown in FIG. 11 to 13.

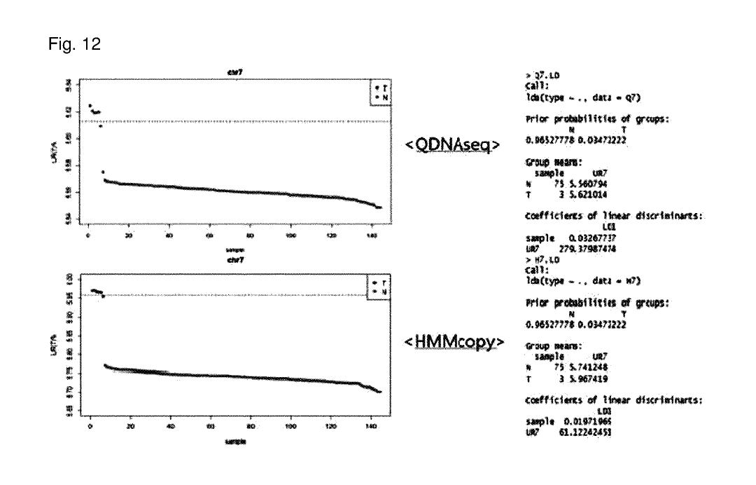

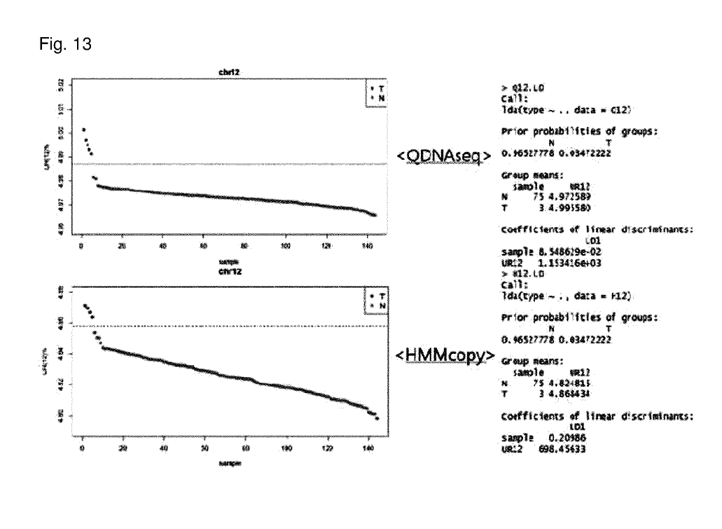

In FIG. 11 to 13, it can be confirmed that the same ratio is obtained by defining a minimum number of reads by analyzing the aneuploidic and normal samples of chr13, chr18, and chr21. When the chromosome abnormalities are determined by the LDA according to the present invention with respect to the chromosomes chr3, chr7, and chr12 which are randomly selected by applying the minimum read number, it was confirmed that the normal and the aneuploidy are clearly discriminated as shown in chr3 (FIG. 11), chr7 (FIG. 12) and chr12 (FIG. 13).

In FIG. 11, when an average value of normal samples of chr3 is confirmed by applying the loess algorithm provided by QDNAseq, it is confirmed that the average value is 7.55 and the maximum value is 7.58 and thus, the two values are clearly discriminated from the minimum value of the aneuploidic sample of 7.62.

In FIG. 12, it can be seen that an average value of the normal samples of chr7 is 7.29 and an average value of the aneuploidic samples is 7.36 by applying HMMcopy. It can be seen that even when the minimum value is applied, all the five samples are clearly discriminated from the normal, and as a result, the target chromosome of the method for determining the chromosome abnormalities of the present invention can be extended to all chromosomes.

In FIG. 13, it can be seen that even in the case of chr12, when using QDNAseq, the average of the normal samples is 4.97 and the average value of the aneuploidic samples is 4.995, which are clearly discriminated, and the two values are discriminated with the distance from the maximum value. Even in the case of the HMM copy, it can be seen that the average value of the normal samples is 4.82, and the average value of the aneuploidic samples is 4.868, in which there is a difference and a clear threshold line.

It can be seen that in a total of six examples of three chromosomes (chr13, chr18, and chr21) and chr3, chr7, and chr12 among 22 autosomes, the normal and the aneuploidic samples are clearly discriminated. As a result, it can be seen that it is possible to extend the method for determining the chromosome abnormalities according to the present invention to all chromosomes.

### EXAMPLE 4

### Determination of Sex-Chromosome Abnormalities

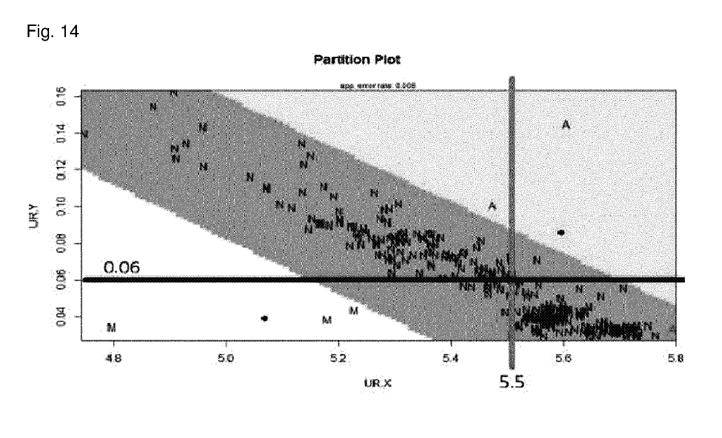

With respect to 246 samples, UR.X and UR.Y indicated by the following Formulas were obtained, and the results were shown in FIGS. 14 to 16.

UR(x) %=Number of reads of chromosome X (chrX)/total number of (autosomes) reads×100

UR(y) %=Number of reads of chromosome Y (chrY)/total number of (autosomes) reads×100

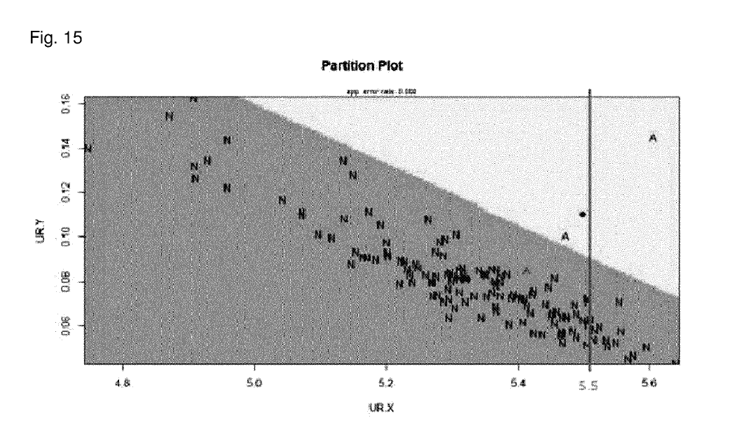

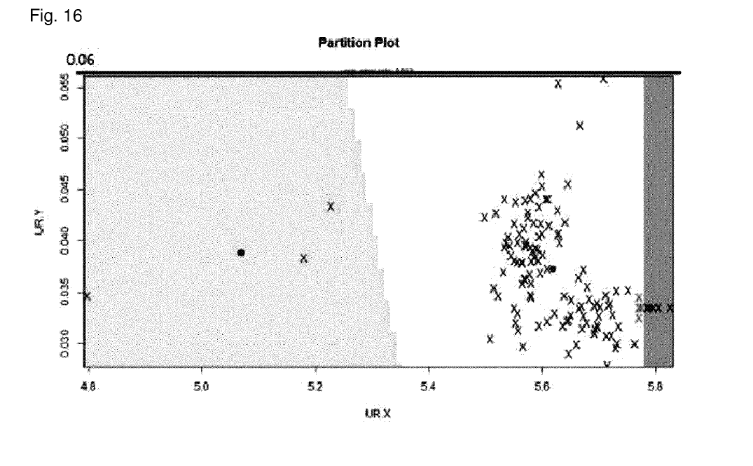

In FIG. 14, the blue and pink portions are set as threshold lines to discriminate normal and aneuploidic samples, and even in the case of a male sample, as shown in FIG. 15, when the value of UR.X is 5.5 or more, it is indicated as XXY, and when the value of UR.X is less than 5.5, it is indicated as XYY. In the case of a female sample, as shown in FIG. 16, a white portion indicates XO and data of 5.75 or more (red A) is determined as XXX.

In the case of the male sample, as shown in FIG. 15, in the case of XO, a value of UR.X of 5.35 or less and UR.Y of 0.06 or less is set to XO, and a threshold line is set along the sky blue line of XO.

When a lot of data is accumulated, more learning is performed, so it is possible to catch a more precise threshold line, and it is possible to obtain a much higher accuracy than the related art because the threshold line can be caught according to the data type.

The results of determining chromosome abnormalities of autosomes and sex-chromosomes by the method for determining the chromosome abnormalities according to present invention are shown in Table 2 below. It can be seen that the results verified by the existing known standard experimental methods and the results determined by the method for determining the chromosome abnormalities according to present invention are the same as each other.

TABLE 2

| | | | | | | | Abnormal |
|---|---|---|---|---|---|---|---|
| | | Male | | Female | | Total | rate |
| Normal | | | 111 | | 95 | 206 | 100% |
| Abnormal | Trisomy 13 | | 3 | | 1 | 4 | 100% |
| | Trisomy 18 | | 3 | | 5 | 8 | 100% |
| | Trisorrry 21 | | 12 | | 10 | 22 | 100% |
| | SCA | XXY | 1 | XXX | 1 | 6 | 100% |
| | | XYY | 1 | XO | 3 | | |

| | | | | |
|---|---|---|---|---|
| Total | 131 | 115 | 246 | 100% |

### INDUSTRIAL AVAILABILITY

The method for determining the chromosome abnormalities according to the present invention is not limited to the sequencing method and the normalization method thereof by a specific automatic sequencing device in the related art. The method can be usefully used for prenatal diagnosis by using the generated sequence information, being applied to autosomes and sex-chromosomes, and early determining presence or absence of malformation due to abnormality of the number of fetal autosomes and sex-chromosomes based on a commercial application of a non-invasive method because as the number of diagnoses increases, accuracy and sensitivity increase.

In the method according to the present invention, when many sequencing data and abnormality determination data therefor are accumulated, it is possible to set a precise threshold line by a linear discriminant analysis (LDA) method, thereby obtaining the sensitivity much higher than that of the conventional method.

1. A method for determining chromosome abnormalities comprising:
a first step of extracting a unique read from sequenced sequencing data of a target chromosome;
a second step of setting a threshold line for determining chromosome aneuploidy by linear discriminant analysis (LDA) by dividing and labeling normality and aneuploidy of chromosome data pre-verified for the normality and aneuploidy; and
a third step of determining whether there is aneuploidy of the unique read-target chromosome gene extracted in the first step by the threshold line set in the second step.

2. The method for determining chromosome abnormalities of claim 1, wherein in the second step of performing initial learning by the LDA method by discriminant-labeling the normality and the aneuploidy of the pre-verified chromosome data and setting the threshold line for determining chromosome aneuploidy, a minimum value of the aneuploidy chromosome data among the pre-verified chromosome data is set as the threshold line.

3. The method for determining chromosome abnormalities of claim 1, wherein in the step of extracting the unique read, the read which is divided into a 90 kb bin region and has the GC content of 0.35 to 0.55 or less is extracted.

4. The method for determining chromosome abnormalities of claim 1, wherein the chromosome is at least one chromosome selected from the group consisting of chromosome 13, chromosome 18, chromosome 21, chromosome 3, chromosome 7, and chromosome 12, a chromosome X or a chromosome Y.

5. The method for determining chromosome abnormalities of claim 1, further comprising:
after the first step, a 1-1 step of calculating UR(x) % (percentage of reads uniquely matched to a chromosome X) and UR(y) % (percentage of reads uniquely matched to a chromosome Y) represented by the following Formulas from the extracted unique read;
UR(x) %=Number of reads of chromosome X (chrX)/total number of (autosomes) reads×100
UR(y) %=Number of reads of chromosome Y (chrY)/total number of (autosomes) reads×100
a 1-2 step of discriminating gender from the UR(x) % and the UR(y) %; and
a 1-3 step of discriminating gender from the number of reads of the region matched to a Y-specific region in the step of discriminating the gender from the UR(x) % and the UR(y) %.

6. The method for determining chromosome abnormalities of claim 4, wherein when the target chromosome is a chromosome X, the chromosome abnormalities are determined as XXX or XO.

7. The method for determining chromosome abnormalities of claim 4, wherein when the target chromosome is a chromosome Y, the chromosome abnormalities are determined as XXY or XYY.

8. The method for determining chromosome abnormalities of claim 1, wherein the first to third steps are repeated N times.

9. The method for determining chromosome abnormalities of claim 8, wherein the determination of the aneuploidy for a chromosome data Dn used at the time of the N-th determination is a threshold value derived from a chromosome data Dn−1 used at the time of the N−1-th determination.

10. The method for determining chromosome abnormalities of claim 1, wherein the sequenced sequence data is obtained by a next-generation sequencing platform.

11. The method for determining chromosome abnormalities claim 1, wherein the sequenced sequence data is obtained by a sequencing platform including the use of a polymerase chain reaction.

12. The method for determining chromosome abnormalities claim 1, wherein the sequenced sequence data is obtained by a sequencing platform including the use of sequencing by synthesis.

13. The method for determining chromosome abnormalities claim 1, wherein the sequenced sequence data is obtained by a sequencing platform including the use of ions, for example, hydrogen ion release.

14. The method for determining chromosome abnormalities claim 1, wherein the sequenced sequence data is obtained by a sequencing platform including the use of a semiconductor-based sequencing method.

15. The method for determining chromosome abnormalities claim 1, wherein the sequenced sequence data is obtained by a sequencing platform including the use of a nanopore-based sequencing method.

16. The method for determining chromosome abnormalities of claim 10, wherein the next-generation sequencing platform is selected from a Roche 454 (i.e., Roche 454 GS FLX), a SOLiD system from Applied Biosystems (i.e., SOLiDv4), GAIIx, HiSeq 2500 and MiSeq sequencers from Illumina, Ion Torrent semiconductor sequencing platforms from Life Technologies, PacBio RS from Pacific Biosciences, and 3730xl from Sanger.

17. The method for determining chromosome abnormalities claim 1, wherein the sequenced sequence data is obtained by an Ion Torrent platform from Life Technologies or MiSeq from Illumina.

18. The method for determining chromosome abnormalities claim 1, wherein the sequenced sequence data is obtained by an Ion Torrent personal genome machine (Ion Torrent PGM) from Life Technologies.

19. The method for determining chromosome abnormalities claim 1, wherein the sequenced sequence data is obtained by multiplex capable iteration based on an Ion Torrent platform from Life Technologies, Ion Proton having PI or PII chips, S5 and its further derivative devices and components thereof.

20. The method for determining chromosome abnormalities claim 1, wherein the sequenced sequence data is normalized or not.
